(19) United States
(12) Patent Application Publication
(10) Pub. No.: US 2018/0221052 A1
(43) Pub. Date: Aug. 9, 2018

(54) ANTI-FOULING CANNULAS FOR ENDOSCOPIC PORTS

(71) Applicant: Galen Ohnmacht, Crownsville, MD (US)

(72) Inventor: Galen Ohnmacht, Crownsville, MD (US)

(21) Appl. No.: 15/887,583

(22) Filed: Feb. 2, 2018

Related U.S. Application Data
(60) Provisional application No. 62/453,783, filed on Feb. 2, 2017.

(51) Int. Cl. A61B 17/34; A61L 29/14; A61L 29/06

(52) U.S. Cl. CPC A61B 17/3423; A61L 29/14; A61B 2017/00867; A61L 2400/16; A61L 29/06

(57) ABSTRACT

A cannula incorporating a blood wicking/diversion mechanism to absorb and/or obstruct blood from running down the cannula onto an endoscope inserted therein, and/or the procedure site, thereby obfuscating the field-of-view during a thoracic procedure. In an embodiment the device comprises an elongate woven fabric wick contained within an open-faced lumen molded into the bottom wall of the cannula tubular body and running along its length to absorb blood or fluid running down its length. In another embodiment the wick is formed in a sleeve-like configuration slightly larger in diameter than tubular body (configured for slidable insertion thereon) woven back into a string at the distal end. Alternatively the device comprises an expandable dome-shaped hood attached to the cannula.

### CROSS-REFERENCE TO RELATED APPLICATION(S)

The present application derives priority from U.S. Provisional Patent Application 62/453,783 filed 2 Feb. 2017.

### BACKGROUND OF THE INVENTION

### 1. Field of the Invention

The present invention relates generally to endoscopic surgery, and more specifically, to an improved cannula for endoscopic surgery (and particularly thoracic surgery) which facilitates blood absorption and/or diversion during surgery thereby helping to maintain an adequate field-of-view through the endoscope inserted there through.

### 2. Description of the Background

Many surgical procedures entail remote visualization of a surgical site. For example, thoracic surgical procedures are currently performed to investigate, diagnose, and treat diseases of the heart and the vessels of the thorax. Such procedures include repair and replacement of mitral, aortic, and other heart valves, repair of atrial and ventricular septal defects, pulmonary thrombectomy, treatment of aneurysms, electrophysiological mapping, ablation of the myocardium, and others. In the past thoracic procedures inevitably began with a gross thoracotomy, usually a median sternotomy, to gain access into the patient's thoracic cavity. A large opening into the thoracic cavity is thus created, through which the surgical team may directly visualize and operate upon the heart and other thoracic contents.

Today there exist minimally invasive alternatives utilizing endoscopic devices and instruments which are introduced within the thoracic cavity via ports or trocars positioned within the intercostal spaces. Endoscopes are well-known medical devices that use long flexible fibers for visualizing the interior of a body or body part for diagnostic or therapeutic purposes. For therapeutic endoscopy some actual treatment is carried out via the endoscope, while for diagnostic endoscopy entails visualizing to aid diagnosis.

In the past, endoscopic surgery has been facilitated by the use of trocars and cannulas to create operative surgical ports to gain entrance into the body for insertion and manipulation of the endoscope. Typically, these cannulas employed a thin tube or tubes as the passageway for the endoscope.

As seen in FIG. 1 for thoracic surgery the trocar/cannula must be inserted between the ribs. The ribs consist of highly vascular cancellous tissue, enclosed in a thin layer of compact bone. Thus, any penetration no matter how small will result in bleeding. When an endoscope is then inserted through the cannula for viewing the surgical site, the blood tends to travel down the cannula and onto the endoscope, down the endoscope to the imaging end, onto the lens, eventually dripping onto the surgical site. Blood on the lens of the endoscope will directly blur or block the view, while covering the area that the surgeon wants to see. The residual blood makes it harder to distinguish shapes and colors.

There are existing suction-type and irrigation-type aspirators for removal of blood before it obscures the view of the endoscope lens. These are typically sold as endoscope accessories in the form of a removable sheath that slides over the endoscope, and is connected to a vacuum or irrigation system. Vacuum or irrigation fluid cleans the distal end of the endoscope, thereby clearing or rinsing the lens. However, these aspiration/vacuum systems have a common shortcoming: they require the tip of the endoscope to contact the blood. This does not prevent blood from running down the length of the endoscope and dripping onto the surgical site. The present invention relates to an improved blood wicking-type removal device particularly useful in medical procedures.

Materials and combinations of materials have been proposed to transport or absorb moisture and blood from wounds. For example, U.S. Pat. No. 5,540,964 describes a moisture transport material which utilizes a hydrophilic synthetic material that is employed beneath a cast to reduce moisture conditions, promoting fungal and bacterial growth.

U.S. Pat. Nos. 5,447,505 and 5,977,428 show absorbent dressings for wounds which are intended to permit the removal of wound exudates, including blood, by the use of imbedded hydrogel particles and the predetermination of pore size with a particular polymeric material.

U.S. Pat. No. 5,928,174 shows a wound dressing device in which removal of moisture in the wound area is enhanced by forming absorbent material into a plurality of free floating strands.

U.S. Pat. No. 3,969,498 describes a wound dressing having a water-soluble body which may be aerated and foamed to adhere to wound tissue and form an artificial eschar or a scab.

U.S. Pat. No. 5,466,231 shows a laminated sponge device in which a polyvinyl acetate sponge body is laminated on multiple sides by a surface layer having perforations of a small pore size to permit fluids to pass through the laminate and be absorbed by the sponge body.

What is needed is a blood absorption/diversion device for thoracic cannula that employs a wicking element that is not clogged by clotting blood, thereby helping to maintain an adequate field-of-view through the endoscope inserted there through.

### SUMMARY OF THE INVENTION

These and other objects are accomplished herein by blood wicking and/or diversion mechanisms for cannulas, and specifically for thoracic cannulas, that prevents blood from running down the cannula onto the endoscope and/or procedure site during a thoracic procedure, thereby helping to maintain an adequate field-of-view through the endoscope inserted there through.

In an embodiment, the blood wicking/diversion mechanism comprises an elongate woven fabric wick contained within an open-faced lumen molded into the bottom wall of the cannula tubular body and running along its length. The exposed wick absorbs blood or fluid running down its length. In another embodiment the wick is formed in a sleeve-like configuration slightly larger in diameter than tubular body (configured for slidable insertion thereon), and woven back into a string at the distal end that arches downward. In yet another embodiment, a blood diversion mechanism comprises an expandable dome-shaped hood attached to the cannula.

All the foregoing alone or in combination wick and/or obstruct blood from running down the cannula onto the endoscope and/or procedure site during a thoracic procedure, thereby maintaining a clear field-of-view through the endoscope inserted there through.

### BRIEF DESCRIPTION OF THE DRAWINGS

Other objects, features, and advantages of the present invention will become more apparent from the following detailed description of the preferred embodiments and certain modifications thereof when taken together with the accompanying drawing in which:

FIG. 1 is a perspective drawing of thoracic post-surgery with cannula inserted between the ribs.

FIG. 2 is a perspective illustration of the thoracic cannula 2 with trocar 20 according to an embodiment of the invention.

FIG. 3 is a close-up side perspective illustration of the tip of thoracic cannula 2 with exemplary blood diversion mechanism 30.

FIG. 4 is a close-up front perspective illustration of the tip of thoracic cannula 2 with exemplary blood diversion mechanism 30 as in FIG. 3.

FIG. 5 is a side perspective view of an alternative embodiment of the blood wicking/diversion mechanism 130.

FIG. 6 is a side perspective view of yet another blood diversion mechanism 40 that may be used alone or in combination with blood wicking/diversion mechanisms 30, 130.

### DETAILED DESCRIPTION OF THE PREFERRED EMBODIMENTS

The present invention is a cannula incorporating an integral blood wicking and diversion mechanism and, specifically, a thoracic cannula for passing an endoscope that incorporates a blood wicking/diversion mechanism that prevents blood from running down the cannula onto the endoscope and/or procedure site during a thoracic procedure, thereby helping to maintain an adequate field-of-view through the endoscope inserted there through.

As shown in FIG. 2, the thoracic cannula 2 generally comprises an elongated hollow tubular body 10 having an open distal end and an open proximal end provided with a flat, annular flange 12 extending radially outward from the tubular body 10. Cannula 2 may be formed of clear plastic material which is flexible and resilient, e.g., polyurethane. The material and wall thickness of the cannula 2 are preferably selected such that the tubular body 10 is somewhat flexible without permanent distortion. The tubular body 10 has a relatively small inner diameter, e.g., 10 mm, which is small enough to fit between the adjacent ribs in an intercostal space of the body wall.

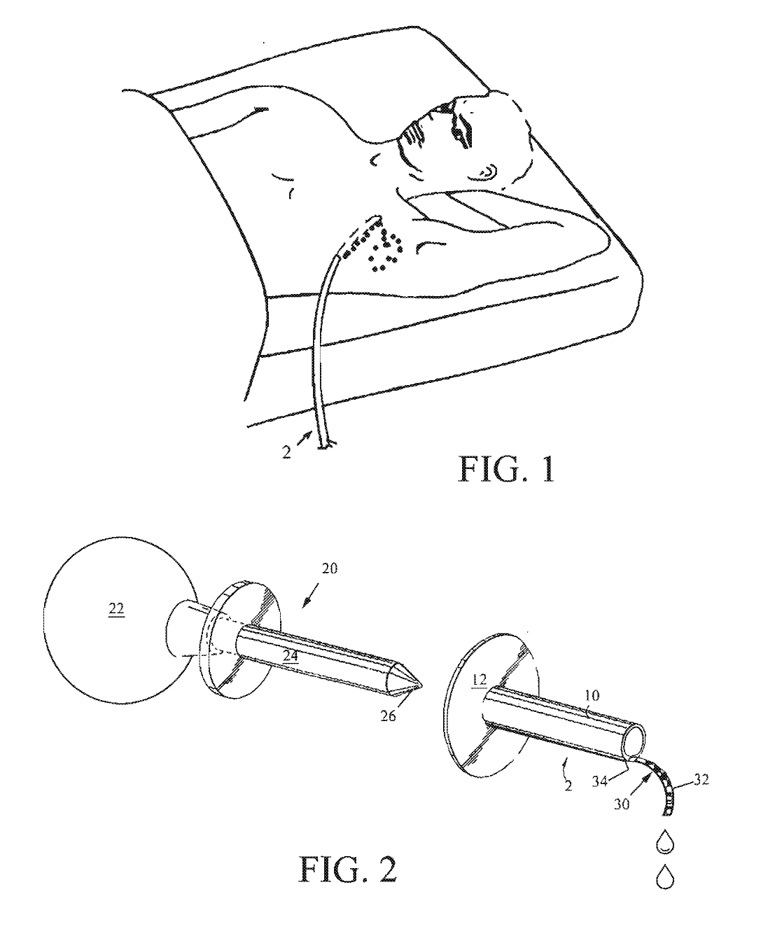

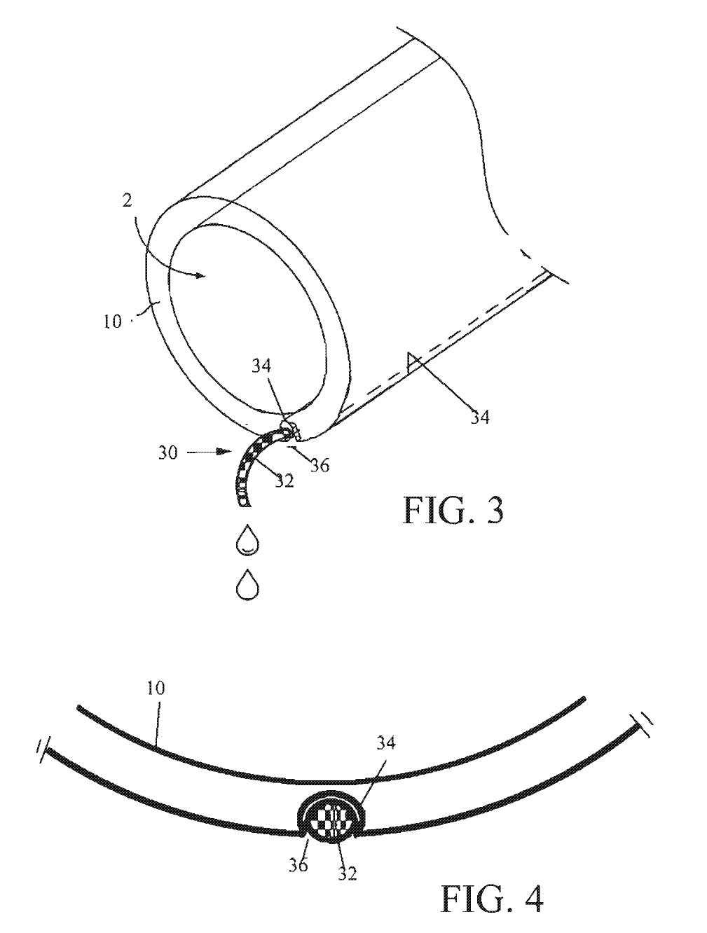

A trocar 20 may be used to install cannula 2 into a body wall at an intercostal space between a pair of adjacent ribs, as per FIG. 1. The trocar 20 may include a trocar handle 22 which supports an elongated, cylindrical obturator 24 with a pointed distal end 26. The trocar 20 is fully inserted into the cannula 10 such that the obturator 26 extends through the tubular body 10 and the pointed end 26 extends outward of tubular body 10. This way, the cannula 2 may be inserted on trocar 20 via its pointed end 26 through an intercostal incision until the annular flange 12 engages the external surface of the body wall. Then the trocar 20 is pulled outward to remove it from cannula 2, leaving cannula 2 in place to serve as a surgical port that enables surgical instruments, e.g. an endoscope, to be inserted and manipulated within the thoracic cavity.

In accordance with the invention, the cannula 2 incorporates a blood wicking/diversion mechanism 30 that prevents blood from running down the cannula onto the endoscope and/or procedure site during a thoracic procedure, thereby helping to maintain an adequate field-of-view through the endoscope inserted there through. Blood wicking/diversion mechanism 30 comprises an elongate woven fabric wick 32 contained within an open-faced lumen 34 both of which run along the bottommost surface of the tubular body 10. The open-faced lumen 34 is molded into the bottom wall of the tubular body 10 and preferably passes entirely and uniformly through the entire cannula 2, exiting on one side opposite the annular flange 12 and on the other side at the distal end of the tubular body 10. The open-faced lumen 34 has smooth internal walls defined by a uniform circular cross-section interrupted by a longitudinal absorption slot 36. Importantly, the longitudinal absorption slot 36 lies along a chord that cuts off a circular segment ( [symbol] ) of the circular cross-section of the interior of tubular body 10, and the circular segment ( [symbol] ) is less than 180°, more preferably within a range of from 160 to 40 degrees, and most preferably about 90 degrees. The elongate woven fabric wick 32 occupies a majority of the lumen 34 and is sized accordingly to be contained therein, too thick to pass through slot 36, and effectively trapped within lumen 34 yet frontally exposed through slot 36. This leaves wick 32 free to absorb blood or fluid running down its length. The woven fabric wick 32 preferably remains slidable within lumen 34 and may be removed, if desired, or extended from exterior of the body. In is normal position the woven fabric wick 32 extends outward from lumen 34 at the distal tip of cannula 2 and arcs downward under natural force of gravity approximately an inch or two. This way, when cannula 2 is used as a surgical port for an endoscope and the endoscope protrudes through cannula 2 into the thoracic cavity, any residual blood or fluid running down cannula 2 under force of gravity will either be absorbed into the woven fiber of the wick 32 or directed down the wick 32 away from the endoscopic lens and/or surgical site, thereby helping to maintain a clear field-of-view through the endoscope.

One skilled in the art will appreciate that the woven fabric wick 32 may be carried in external collars, sleeve(s) or eyelets attached to tubular member 10 rather than an internal lumen 34 without departing from the scope of spirit of the invention.

In addition, the wick 32 itself may be formed in a sleeve-like configuration to eliminate the need for lumen 34.

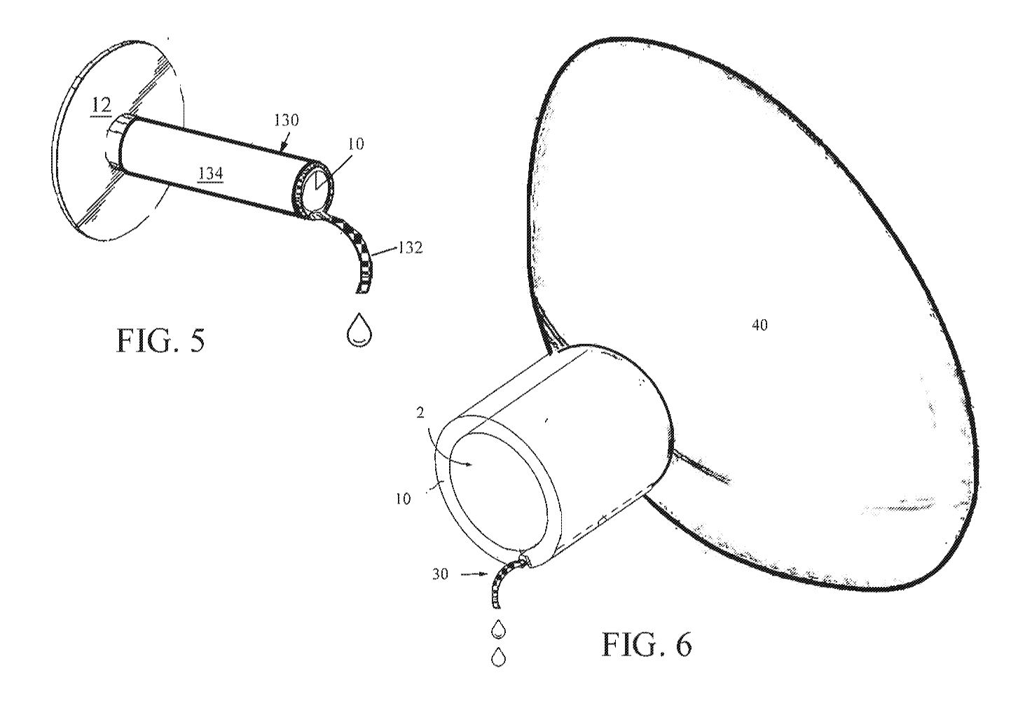

For example, FIG. 5 illustrates an alternative embodiment in which the blood wicking/diversion mechanism 130 comprises a tubular plastic sleeve 134 slightly larger in diameter than tubular body 10 and configured for slidable insertion thereon, and an elongate tubular woven fabric wick 132 contained within sleeve 134. The wick 132 is woven in a tubular weave along its length to conform to the tubular body 10 but, as illustrated, is woven back into a string at the distal end of the tubular weave. This allows wick 132 to arch downward as described previously. Just as above, the blood wicking/diversion mechanism 130 prevents blood from running down the cannula onto the endoscope and/or procedure site during a thoracic procedure, thereby helping to maintain an adequate field-of-view through the endoscope inserted there through.

FIG. 6 illustrates yet another blood diversion mechanism 40 that may be used alone or in combination with blood wicking/diversion mechanisms 30, 130. Blood diversion mechanism 40 comprises an expandable dome-shaped hood attached to cannula 2. The hood 40 may be made of silicone hydrogel in thickness and dimensions similar to a soft contact lens, such that it easily collapses upon insertion of tubular member 10 through the body wall but expands once inside the thoracic cavity. Rather than wicking blood and fluid, hood 40 obstructs blood from running down the cannula 2 onto the endoscope and/or procedure site during a thoracic procedure, again maintaining a clear field-of-view through the endoscope inserted there through.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

I claim:

1. A thoracic surgical apparatus, comprising:
an elongated hollow tubular cannula having a sidewall leading from an open distal end to an open proximal end; an lumen traversing said sidewall from said distal end to said proximal end; and
an absorbent wick inserted in said lumen for absorbing bodily fluid and preventing it from running down the cannula or dripping during a thoracic procedure.

2. The thoracic surgical apparatus according to claim 1, wherein said hollow tubular cannula has a central axis and said wick is coaxial to said central axis.

3. The thoracic surgical apparatus according to claim 1, wherein said cannula comprises a flat, annular flange at said proximal end.

4. The thoracic surgical apparatus according to claim 1, wherein said cannula comprises clear plastic material.

5. The thoracic surgical apparatus according to claim 4, wherein said clear plastic material is flexible with a shape memory characteristic.

6. The thoracic surgical apparatus according to claim 1, wherein said absorbent wick comprises woven fabric.

7. The thoracic surgical apparatus according to claim 1, wherein said lumen is molded into the sidewall of the cannula.

8. The thoracic surgical apparatus according to claim 7, wherein said molded lumen is defined by a uniform semi-circular cross-section.

9. The thoracic surgical apparatus according to claim 8 wherein said semi-circular cross-section is interrupted by an absorption slot open outward.

10. The thoracic surgical apparatus according to claim 9 wherein a width of said absorption slot is less than a diameter of said wick.

11. The thoracic surgical apparatus according to claim 9 wherein a width of said absorption slot is less than a maximum diameter of said lumen.

12. The thoracic surgical apparatus according to claim 11 wherein said semi-circular lumen encloses a circular segment within a range of from 160 to 40 degrees.

13. The thoracic surgical apparatus according to claim 12 wherein said semi-circular lumen encloses a circular segment of 90 degrees.

14. The thoracic surgical apparatus according to claim 10 wherein said wick remains exposed through said absorption slot when inserted in said lumen.

15. The thoracic surgical apparatus according to claim 14 wherein said wick is slidable along said lumen.

16. The thoracic surgical apparatus according to claim 15 wherein said wick is slidably removable from said lumen.

17. A thoracic surgical apparatus, comprising:
an elongated hollow tubular cannula having a sidewall leading from an open distal end to an open proximal end; and
an absorbent wick surrounding the proximal end of said cannula to prevent body fluid from dripping during a thoracic procedure.

18. A thoracic surgical apparatus, comprising:
an elongated hollow tubular cannula having a sidewall leading from an open distal end to an open proximal end; an lumen traversing said sidewall from said distal end to said proximal end;
an absorbent wick inserted in said lumen for absorbing bodily fluid and preventing it from running down the cannula or dripping during a thoracic procedure; and
a trocar for installing said cannula through a patient's intercostal space, said trocar including a handle attached to an elongate cylindrical obturator having a pointed distal end.

19. The thoracic surgical apparatus according to claim 18, wherein said molded lumen is defined by a uniform semi-circular cross-section and said semi-circular cross-section is interrupted by an absorption slot open outward.

20. The thoracic surgical apparatus according to claim 19, wherein said wick is slidably removable from said molded lumen.
